(19) United States
(12) Patent Application Publication
(10) Pub. No.: US 2006/0194895 A1
(43) Pub. Date: Aug. 31, 2006

(54) Dental compositions having dual dental diagnostic capabilities and methods of use

(76) Inventors: Dan Loveridge, Sandy, UT (US); Peter M. Allred, Riverton, UT (US); Neil T. Jessop, Sandy, UT (US)

Correspondence Address: Rick D. Nydegger;WORKMAN NYDEGGER, 1000 Eagle Gate Tower, 60 East South Temple, Salt Lake City, UT, 84111, US

(21) Appl. No.: 11/065,747

(22) Filed: Feb. 25, 2005

(51) Int. Cl. A61K 6/083

(52) U.S. Cl. 523/115

(57) ABSTRACT

A dental composition capable of being used in dual dental diagnostics can include at least one polymerizable resin and at least two different types of color changing materials. Alternatively, a two-part composition can include at least one dental composition configured for placement onto a person's tooth, and at least two different types of color changing materials to be mixed into the at least one dental composition to form a dental composition capable of being used in dual dental diagnostics. The dental composition can be formulated to blend with a person's tooth, and also be capable of changing color to be distinguishable from the tooth. The dental composition can be used in a method for performing a dental procedure by applying the dental composition to a tooth, and exposing the dental composition to at least one color changing stimulus. Accordingly, the dual dental diagnostic capable composition can aid a dental professional in distinguishing between the location of the dental composition and the tooth.

### CROSS-REFERENCE TO RELATED APPLICATIONS

This United States Patent Application cross-references two other United States Patent Applications filed simultaneously herewith on Feb. 25, 2005, entitled, respectively, DENTAL COMPOSITIONS HAVING A PHOSPHORESCENT MATERIAL AND METHODS OF USE with Dan Loveridge as inventor, attorney Docket No. 7678.914, Express Mail Label No. EV462385942US; and DENTAL COMPOSITIONS HAVING ANTI-STOKES UP CONVERTERS AND METHODS OF USE with Dan Loveridge as inventor, attorney Docket No. 7678.917, Express Mail Label No. EV462385868US, which applications are incorporated herein in their entirety.

### BACKGROUND OF THE INVENTION

1. The Field of the Invention

The present invention relates to dental compositions that include dual color changing substances. More particularly, the present invention relates to combining at least two color changing substances into sealants, varnishes, restoratives, adhesives, bonding agents, cements, composites, veneers, and crowns for use in dual dental

2. The Relevant Technology

Teeth are an anatomical feature prone to being easily broken, fractured, discolored, or otherwise damaged. Unlike bones, which can automatically regenerate to correct breaks, fractures or other damage, teeth do not self correct. As such, people rely on dental professionals to repair their teeth, or to enhance the visual characteristics of their teeth.

In order to repair a damaged tooth, a dental professional may apply a dental composition to the tooth. Originally, when a cavity needed to be fixed, the filing was a metallic or blackish color that contrasted with the color of the teeth. Also, older crowns and other dental prosthetics were not designed to precisely match or blend with a patient's teeth. However, in developed societies the aesthetic qualities of teeth have become important socioeconomic as well as physical attractiveness indicators. Thus, colored dental compositions or prosthetics that do not match or blend with a patient's teeth have lost acceptance with the general public.

In response, dental professional now frequently use dental compositions that can blend with a patient's teeth. For example, when a patient has a chipped front tooth, a dental professional can restore the tooth so that it is extremely difficult to discern the boundary between the dental restoration and the natural tooth. Additionally, cavity filling materials have been formulated to blend with teeth. Also, dental compositions have been formulated to enhance the aesthetic qualities to discolored teeth. Thus, when a person needs to fix or improve their teeth, the resulting restoration can blend with their surrounding teeth.

While dental patients have been pleased with the improved aesthetic qualities of recent dental restorations, some dental professionals have become frustrated with the teeth-blending dental compositions. In part, this frustration has resulted from dental restorations that needed to be modified, repaired, removed, and/or replaced at some point after the restoration was performed. When this happens, the dental professionals have also had a difficult time discerning the boundary of the dental restoration from the natural tooth. As a consequence, the dental professional can mistakenly leave the older restoration material on the teeth, or accidentally remove too much natural tooth. The partial removal of the natural tooth can have long term negative consequences, and most dental professionals would prefer to not remove portions of the natural tooth, if possible, when they are working on teeth. Accordingly, there needs to be a balance between the patient's aesthetic needs and the dental professional's need for distinguishing between a natural tooth and a dental restoration.

Therefore, what is needed is an improved dental composition that can be formulated to visually blend with a tooth and surrounding teeth, and which can temporarily change colors in response to a specific stimulus so that a dental professional can distinguish between the teeth and dental restoration.

### BRIEF SUMMARY OF EMBODIMENTS OF THE INVENTION

Generally, an embodiment of a dental composition in accordance with the present invention is capable of blending with a color of a person's tooth. The dental composition can include at least one polymerizable resin and be configured for placement on a person's tooth. Additionally, at least two different types of color changing materials are admixed into the dental composition. Accordingly, the dental composition is capable of a first reversible color change when exposed to a first color changing stimulus, and capable of a second reversible color change when exposed to a second color changing stimulus. Such changes in the color of the dental composition can be used in dental diagnostics to provide alternate color changing pathways so that the dental composition can be distinguished from the natural tooth.

Another embodiment of the present invention is a two-part composition for use in making a dual color changing dental composition that is capable of blending with a color of a patient's tooth. The two-part composition includes at least one dental composition configured for placement onto a person's tooth, and at least two different types of color changing materials for mixing with the at least one dental composition. As such, the admixed dental composition is capable of a first reversible color change when exposed to a first color changing stimulus, and capable of a second reversible color change when exposed to a second color changing stimulus.

A method of performing a dental procedure with a dual color changing dental composition is also in accordance with the present invention. The method includes applying a dental composition to a tooth, where the dental composition includes at least one polymerizable resin and at least two different types of color changing materials. The dental composition is formulated to be capable of blending with the tooth. The method also includes exposing the dental composition to a first color changing stimulus to cause a first reversible change in the color of the dental composition. Additionally, the dental composition can be exposed to a second color changing stimulus to cause a second color change.

These and other advantages and features of the present invention will become more fully apparent from the following description and appended claims, or may be learned by the practice of the invention as set forth hereinafter.

### BRIEF DESCRIPTION OF THE DRAWINGS

To further clarify the above and other advantages and features of the present invention, a more particular description of the invention will be rendered by reference to specific embodiments thereof which are illustrated in the appended drawings. It is appreciated that these drawings depict only typical embodiments of the invention and are therefore not to be considered limiting of its scope. The invention will be described and explained with additional specificity and detail through the use of the accompanying drawings in which:

FIG. 1A is a side view of a tooth with a cavity that is filled with a dual color changing filling;

FIG. 1B is a top view of the tooth in FIG. 1A;

FIG. 2A is a side view of a tooth with a cavity that is filled with a dual color changing filling;

FIG. 2B is a top view of the tooth in FIG. 2A;

FIG. 3A is a top view of a tooth with a pit and fissure;

FIG. 3B is a top view of the tooth in FIG. 3A with the pit and fissure restored with a dual color changing sealant;

FIG. 4A is a side view of an embodiment of a dental procedure where a crown is being placed onto a tooth with a dual color changing prosthetic adhesive;

FIG. 4B is a side view of an embodiment of a dental procedure where a crown is affixed to a tooth;

FIGS. 5A and 5B are side views of an orthodontic bracket adhered to a tooth with a dual color changing bracket adhesive; and

FIGS. 6A and 6B are side views of a tooth having a dual color changing dental restorative composition.

### DETAILED DESCRIPTION OF EMBODIMENTS OF THE INVENTION

Generally, embodiments of the present invention include dental compositions, two-part dental compositions, and associated methods of making or using the compositions. The terminology employed herein is used for the purpose of describing particular embodiments only and is not intended to be limiting.

As used herein, the term “color changing” refers to a substance or composition that changes color, becomes luminescent, or changes to a contrasting shade when exposed to a certain stimulus or condition, and returns to the original color after the stimulus is removed. Examples of color changing substances include, without limitation, fluorescent, phosphorescent, photochromic, and thermochromic materials

As used herein, the term “fluorescent” refers to any substance that becomes luminescent when exposed to light. Typically, a fluorescent material can appear to slightly change color or emit color more brightly while being exposed to a UV-light stimulus, and cease to be luminescent when the UV-light is removed.

As used herein, the term “photochromic” refers to any substance that can change colors or become a contrasting shade when exposed to light. For example, a photochromic substance can originally appear to be clear or white, but darken to a grey color when stimulated by being irradiated with light. A photochromic substance will return to the original color after it is no longer being irradiated with light.

As used herein, the term “thermochromic” refers to any substance that can change color in response to heating or cooling. For example, a thermochromic substance may appear to have an original color or no color under a normal temperature, but change to emit a different color when stimulated by either being heated or cooled. A thermochromic substance will return to the original color after it returns to the normal temperature.

As used herein, the term “phosphorescent” refers to any substance containing electrons which temporarily enter an excited state upon exposure to a source of energy, such as light, electricity, or heat, and emit photons of visible light as they return to an unexcited state thereby creating a luminescence that is still visible after the source of energy has been removed. As such, phosphorescent materials can glow in the dark.

As used herein, the term “dispersement” or “dispersing” refers to placing and distributing various color changing-materials within a composition.

As used herein, the term “dispersing agent” refers to any substance that prevents color changing materials (particles) from agglomerating or otherwise settling out of a composition.

As used herein, the term “effective amount” refers to at least the minimal amount of a substance or agent, which is sufficient to achieve a desired effect. For example, an effective amount of a color changing material in a dental composition would be the minimum amount that provides the desired color change so that a dental v z professional can distinguish between the dental composition and the surrounding teeth.

As used herein, the term “white light” refers to broad spectrum light that can range from IR-light to UV-light. For example, the light emitted from the sun and regular light bulbs can be considered white light.

As used herein, the term “normal conditions” refers to the ordinary conditions of temperature and light that exist in a mouth when it is open or closed in routine daily activities that commonly occur. For example, the opening and closing of a mouth during a conversation or a smile can be characteristic of “normal conditions.”

It should be recognized that the chemical nomenclature employed herein is used for convenience and brevity, and any of the elements recited additionally includes the monovalent or multivalent ions thereof. For example, europium (Eu) includes the element and the monovalent, divalent, trivalent, and other multivalent europium ions, if the valence is possible.

Concentrations, amounts, particles sizes and other numerical data may be presented in a range format. It is to be understood that such a range format is used merely for convenience and brevity and should be interpreted flexibly to include not only the numerical values explicitly recited as the limits of the ranges, but also to include all the individual numerical values or sub-ranges encompassed within that range as if each numerical value and sub-range is explicitly recited. For example, the particle diameter range of about 0.5 micrometers to about 50 micrometers should be interpreted to include not only the explicitly recited limits of about 0.5 micrometers and about 50 micrometers, but also to include such individual diameters such as 1, 2, 10, and 20 micrometers as well as sub-ranges between these individual diameters. This interpretation should apply regardless of the breadth of the range or the characteristic being described, and should apply to ranges having both upper and lower numerical values as well as open-ended ranges reciting only one numerical value.

Dental compositions in accordance with embodiments of the present invention can include, for example, sealers, permanent fillings, temporary fillings, cements, varnishes, composites, adhesives, and the like. Accordingly, these dental compositions can be configured for being cured after being placed into the mouth such as for filling and sealing root canals. In one embodiment, dental compositions can be formulated for adhering veneers, inlays, onlays, crowns, pontics, and bridges in the mouth. In another embodiment, the dental compositions can be used in orthodontics for affixing an orthodontic bracket to a tooth.

Generally, a dental composition in accordance with an embodiment of the present invention can be formulated to blend with a person's tooth, and also be capable of reversible dual color changes. The dental composition can include at least one polymerizable resin and at least two different types of color changing materials that are all admixed together into a dental composition that is configured for placement on a person's tooth. The dental composition is formulated to blend with the person's tooth under natural conditions and/or white light. Additionally, the at least two different types of color changing materials enables the dental composition to be capable of a first color change when exposed to a first reversible color changing stimulus, and capable of a second reversible color change when exposed to a second color changing stimulus.

In another embodiment of the present invention, a kit having a combination of compositions can be provided. Such a combination of compositions can include at least one dental composition configured for placement onto a person's tooth, and at least one composition comprised of at least two different types of color changing materials. The combination of compositions can be formulated to blend with the person's tooth under normal conditions and/or white light. Additionally, the combination of compositions can be capable of a first color change when exposed to a first reversible color changing stimulus, and capable of a second reversible color change when exposed to a second color changing stimulus.

Also, a dental composition can include any composition that is formulated to be mixed or added with another composition prior to being fixedly applied to a tooth. As such, the resultant mixture of compositions can be properly formulated to adhere to a tooth, where the individual compositions may or may not have sufficient dental-compatibility or adherence.

Additionally, the dental composition can include solid prefabricated dental prostheses. Accordingly, a dental prosthesis such as a veneer, crown, inlay, onlay, pontic, or bridge can be prefabricated before being adhered to the patient's tooth. These dental prostheses can be comprised of a dental composite that has been cast into a solid form. Additionally, a dental prosthesis can be affixed to a tooth with an adherent dental composition such as a dental composition that is capable of dual dental diagnostics. Alternatively, the dental prosthesis can include at least two different types of color changing materials.

The color changing components can be admixed into a dental composition in an effective amount sufficient for changing color, even if only slightly, when exposed to the stimulus that induces the color change. When the dental composition is exposed to the stimulus, the color changing substance can then emit a color that is distinguishable from the tooth for use in dental diagnostics. Examples of color changing substances that can be included in a dental composition can include fluorescent materials, thermochromic materials, photochromic materials, and phosphorescent materials.

A wide range of fluorescent materials can be included in a dental composition such as organic pigments and inorganic complexes. Accordingly, the present invention is not intended to be limited to certain types of fluorescent materials, but can include any type of material that can appear to be luminescent after being exposed to UV-light. Some examples of fluorescent materials can include, without limitation, coumarin derivatives, phthalimide derivatives, fluoranthene derivatives, perylene derivatives, xanthene derivatives, thioxanthene derivatives, pyrano-benzopyran-2,5-dione derivatives, pyrano-quinoline-2,5 derivatives, pyrazole quinoxaline derivatives, 2-pyrano-isoquinoline-3,6-dione derivatives, benzimidazo-benz-isoquinoline-7-one derivatives, acridine derivatives, and the like. Additionally, rare earth complexes such as lanthanide complexes can be used as fluorescent materials. More particular examples of fluorescent materials can include fluoresceins, eosins, and rhodamines. When used, fluorescent materials can be present, for example, at a preferred range of from about 0.00001% to about 15% by weight, a more preferred range of from about 0.001% to about 10% by weight, and a most preferred range of from about 0.1% to about 5% by weight of the dental composition. However, higher or lower concentrations can be used in some embodiments, which can depend on the type and concentration of other constituents in the composition.

Also, a wide range of photochromic materials can be admixed into a dental composition. As such, any type of photochromic material can be included in the present invention as long as it can be formulated into a dental composition that can blend with id z X t teeth under normal conditions, and change colors when irradiated with light. Some examples of photochromic materials can include spiroxazines, spiropyrans, thiopyrans, benzopyrans, benzothioxanthones, dinitrated spiropyrans, naphthacenequinones, naphthopyran, spiroindolines, naphthoxazines, spiro-benzoxazines, and derivatives thereof. Additional examples, without limitation, include substances based on cis/trans isomerism of azobenzene compounds or stilbenes, on the interconversion or electrocyclic ring-closure/ring-opening reaction of spiropyran systems or spirooxazins, or on the 1,5-electrocyclization of pentadienyl anions. When used, the photochromic materials can be present, for example, at a preferred range of from about 0.0001% to about 15% by weight, more preferred range of from about 0.01% to about 10% by weight, most preferred range of from about 0.1% to about 5% by weight of the dental composition. However, higher or lower concentrations can be used depending on the type and concentration of other constituents in the composition.

Various thermochromic materials can also be included within a dental composition, where the type of-thermochromic material is not limited. As such, any thermochromic material that can be formulated into a dental composition that blends with a patient's teeth under normal oral temperatures can be used so long as the magnitude of the temperature change to effect the color change is not too large. Typically, a thermochromic material includes an electron donor and an electron acceptor pair. Some examples of electron donors include substituted phenylmethanes, fluoranes, indolylphthalides, spiropyranes, coumarins, and the like. Some examples of electron acceptors include phenols, azoles, organic acids, and esters. The amount and type of thermochromic material present in the dental composition can be varied depending on the temperature change desired in order to effect the color change.

More particularly, the electron-donating organic compounds can include the following: (i) fluorans, such as 3-diethylamino-6-methyl-7-chlorofluoran, 3-diethylaminobenzo(a)-fluoran, 3,6-diphenylaminofluoran, 3-amino-5-methylfluoran, 2-methyl-3-amino-6,7-dimethylfluoran, 2-bromo-6-cyclohexylaminofluoran, and the like; (ii) diarylphthalides, such as crystal violet lactone, malachite green lactone, and the like; (iii) polyarylcarbinols, such as crystal violet carbinol, malachite green carbinol, and the like; (iv) leuco auramines, such as N-(2,3-dichlorophenyl)leuco auramine, N-benzoylauramine, N-acetylauramine, and the like; (v) rhodamine beta lactams; (vi) indolines, such as 2-(phenyliminoethylidene)-3,3-dimethylindoline, and the like (vii) spiropyranes, such as N-3,3-trimethylindolinobenzospiropyrane, 8-methoxy-N-3,3-trimethylindolinobenzospiropyrane, and the like. Additionally, the electron-accepting compounds can include the following: (i) phenols, such as bisphenol A, p-phenylphenol, dodecylphenol, o-bromophenol, ethyl p-oxybenzoate, methyl gallate, phenol resin, and the like; (ii) metal salt phenolates including the metals sodium (Na), potassium (K), lithium (Li), calcium (Ca), zinc (Zn), aluminum (Al), magnesium (Mg), nickel (Ni),: cobalt (Co), tin (Sn), copper (Cu), iron (Fe), titanium (Ti), lead (Pb), molybdenum (Mo), and like metal salts of phenol; (iii) aromatic carboxylic acids and C₂-C₅ aliphatic carboxylic acids, such as phthalic acid, benzoic acid, acetic acid, propionic acid, and the like; (iv) metal carboxylates, such as sodium oleate, zinc salicylate, nickel benzoate, and the like; (v) acidic phosphate esters and metal salts thereof, such as butyl acid phosphate, 2-ethylhexyl acid phosphate, dodecyl acid phosphate, diotolyl phosphate, and the like; (vi) triazole compounds, such as 1,2,3-triazole, 1,2,3-benzotriazole, and the like; (vii) thiourea and derivatives thereof, such as diphenylthiourea, diotoluylurea, and the like; (viii) halohydrines, such as 2,2,2-trichloroethanol, 1,1,1-tribromo-2-methyl-2-propanol, N-3-pyridyl-N′-(1-hydroxy-2,2,2-trichloroethyl)urea and the like; (ix) benzothiazoles, such as 2-mercaptobenzothiazole, 2-(4′-morpholinodithio)benzothiazole, N-tert-butyl-2-benzothiazolyl sulfenamide, 2-mercaptobenzothiazole zinc salt, and the like.

Additionally, a wide variety of phosphorescent materials can be utilized in the present invention. Examples of phosphorescent materials can include sulfides, oxides, metal aluminate oxides, rare earth oxides, and other similar phosphors. Additional examples of basic phosphorescent materials can include calcium sulfates (CaS), calcium strontium sulfates (CaSrS), zinc sulfates (ZnS), zinc cadmium sulfates (ZnCdS), barium zinc sulfides (BaZS), barium zinc cadmium sulfides (BaZCdS), and strontium sulfides (SrS). Also, a long-life phosphor can be included, such as a metal aluminate such as an alkaline earth aluminate oxide. These long life phosphors can have the formula MA1204, where the M is an alkaline earth metal or mixture of such metals. Examples of these phosphors include strontium aluminate oxide (SrAl₂O₄), calcium aluminate oxide (CaAl₂O₄), barium aluminate oxide (BaAl₂O₄), and mixtures thereof. When used, the phosphorescent material can be present, for example, at a preferred range of from about 0.0001% to about 15% by weight, more preferred range of from about 0.01% to about 10% by weight, and most preferred range of from about 0.1% to about 5%. However, higher concentrations can be used in some instances.

These phosphorescent materials can additionally include an activator such as aliiminum (Al), silver (Ag), gold (Au), manganese (Mn), bismuth (Bi), gallium (Ga), indium (In), scandium (Sc), terbium (Th), europium (Eu) lanthanum (La), cerium (Ce), praseodymium (Pr), neodymium (Nd), samarium (Sm), gadolinium (Gd), dysprosium (Dy), holmium (Ho), erbium (Er), thulium (Tm), ytterbium (Yb), lutetium (Lu), tin (Sn), bismuth (Bi), or mixtures thereof. The activators are thought, without being bound to theory, to enter the crystal lattice of the host material to impart some phosphorescence thereto.

While certain fluorescent, photochromic, thermochromic, and phosphorescent materials have been described as examples, the present invention is intended to be limited to the listed materials. As such, all currently known and future discovered fluorescent, photochromic, thermochromic, and phosphorescent materials are intended to be capable of being included in the inventive dual color changing dental compositions.

In another embodiment, the color changing materials can be encapsulated in a glass. Accordingly, the color changing materials can be combined with SiO₂ under heat so that glass beads, glass microbeads, or bulk class with color changing characteristics can be formed. For example, the glass can be comprised of silicon (Si), cerium (Ce), antimony (Sb), tin (Sn), zirconium (Zr), strontium (Sr), barium (Ba), aluminum (Al), zinc (Zn), and the like. When formed, the bulk class can then be ground or milled to produce color changing glass particles that can be formulated into the dental composition. It is thought, without being bound to theory, that encapsulating the color changing materials in a glass can increase the longevity of the color changing characteristic, and prevent the color changing materials from leaching out-of the dental composition. Additionally, the glass encapsulated color changing materials can increase biocompatibility.

Similarly, the color changing materials can be encapsulated within a polymeric microbead for much of the same reasons they can be encapsulated in a glass. In one embodiment, the microbead can be comprised of hydrophobic monomers that are polymerized into crosslinked and/or non-crosslinked polymers. Examples of suitable hydrophobic polymers can include crosslinked and non-crosslinked polyacrylates such as polymethyl methacrylate, crosslinked and non-crosslinked polyaromatics such as polystyrene and polyvinyltoluene.

The color changing materials and/or encapsulated materials can also be admixed with a dispersing agent to facilitate the dispersement and retention of the color changing materials in the composition. Examples of dispersing agents include polymers and copolymers of styrene sulfonate salts, acrylic and styrene copolymers, sulfonated polyesters, oleoyl methyl taurine, sodium dodecyl sulfate, amine dispersants, methyl stearate, ethyl stearate, methyl hexanoate, methyl heptanoate, methyl octanoate, methyl laurate, methyl oleate, methyl adipate, methyl caprylate, methyl caproate, methyl anthranilate, methyl palmitate, methyl palmitoleate, methyl oxalate, methyl 2-nonanoate, methyl benzoate, 2-methylbenzophenone, methyl benzilate, methylbenzyl acetate, trimethyl borate, methyl caprate, methyl butyrate, methyl decanoate, methyl cyclohexanecarboxylate, methyl dimethoxyacetate, methyl diphenylacetate, methyl heptanoate, methyl linoleate and the like.

Additionally, the dental compositions can include a variety of materials such as polymerizable resins, polymerization initiators, fillers, coupling agents, plasticizers and the like. The polymerizable resins can include any combination of monomers, oligomers, and polymers having,one or more ethylenically-unsaturated groups, where, ethylenically unsaturated groups can be polymerized by free radial polymerization. Such free radical polymerizable materials include monomers and/or mono-, di- or poly-acrylates and methacrylates. For example, the polymerizable resin can include methyl, acrylate, methyl methacrylate, ethyl acrylate, isopropyl methacrylate, n-hexyl acrylate, stearyl acrylate, allyl acrylate, glycerol diacrylate, glycerol triacrylate, ethyleneglycol diacrylate, diethyleneglycol diacrylate, triethyleneglycol dimethacrylate, diurethane dimethacrylate, 1,3-propanediol diacrylate, 1,3-propanediol dimethacrylate, trimethylolpropane triacrylate, 1,2,4-butanetriol trimethacrylate, 1,4-cyclohexanediol diacrylate, pentaerythritol triacrylate, pentaerythritol tetraacrylate, pentaerythritol tetramethacrylate, sorbitol hexacrylate, the diglycidyl methacrylate of bis-phenol (“Bis-GMA”), bis[1-(2-acryloxy)]-p-ethoxyphenyldimethylmethane, bis[l-(3-acryloxy-2-hydroxy)]-p-propoxyphenyldimethylmehtane, trishydroxyethyl-isocyanurate trimethacrylate, the bis-acrylates and bis-methacrylates of polyethylene glycols of molecular weight 200-500, copolymerizable mixtures of acrylated monomers, and copolymerizable acrylated oligomers, and the like. Alternatively, phosphoric acid derivatives and carboxylic acid derivatives of these ethylenically unsaturated monomers can be used. Also, vinyl compounds such as styrene, diallyl phthalate, divinyl succinate, divinyl adipate and divinylphthalate can be polymerized. However, it should be recognized that this is not an exhaustive listing of polymerizable resins, and other polymerizable resins can be used in accordance with the present invention.

A polymerizable resin can be included in the dental composition over a wide range of concentrations. The concentrations can depend on the amount of filler, plasticizer, and polymerization initiator as well as other factors. For example, the dental composition can have a polymerizable resin such as an ethylenically unsaturated monomer at a preferred range of from about 10% to about 99% by weight, more preferred range of from about 1.5%-80% by weight, and most preferred range of from about 25% to about 50% by weight.

Typically, free radical polymerization requires an initiator to generate a free radical. Various types of initiators can produce a free radical upon being exposed to light, heat, or chemicals. The initiator compounds are provided into the dental compositions of the invention in an effective amount to initiate polymerization or enhance the rate of polymerization or curing.

Photo-initiators are a group of compounds that will generate a free radical when exposed to light having a specific wavelength. As such, different photo-initiators can be selected depending on the wavelength of light that will initiate the polymerization. Examples of photo-initiators can include benzophenone, benzoin, 9,10-phenanthrenequinone, diacetyl, furil, anisil, 4,4′-dichlorobenzil, 4,4′-dialkoxybenzil, phenylpropanedione, acylphosphine oxides, camphorquinone, derivatives thereof, and the like. Photopolymerization can be initiated, for example, by irradiation with light having a wavelength of from about 400 nm to about 500 nm.

Heat-initiators can be used in hot-curing systems, which are particularly suitable for producing dental prostheses such as crowns, inlays, and onlays. Some heat-initiators can be activated with temperatures less than 150° C. Examples of heat-initiators can include t-butyl peroxide, dibenzoyl peroxide, dilauroyl peroxide, t-butyl peroctoate, t-butyl perbenzoate, and the like.

On the other hand, in certain applications a chemical-initiator, which typically is comprised of at least two co-initiators that generate a free radical, can be preferred to induce polymerization. Additionally, a chemical-initiator can at least be used as a co-initiator. These chemical-initiator systems can use a reactive pair, for example, benzoyl peroxide, lauryol peroxide, or dibenzoyl peroxide, in combination with an N,N-dimethyl-p-toluidine, N,N-dihydroxyethyl-p-toluidine, and other similar amines. Alternatively, a combined system including a photo-initiator, heat-initiator, and/or chemical-initiator can be used.

The concentration of the polymerization initiator depends on the concentration of the polymerizable resin, or on the type of initiator. For example, the dental composition can include a polymerization initiator at a preferred range of from about 0.001% to about 5% by weight, more preferred range of from about 0.01% to about 2.5% by weight, and most preferred range of from about 0.1% to about 1% by weight. However, the concentration of initiator can be varied depending on the type of initiator and/or type of resin as well as the desired properties of the composition.

In another embodiment, a dental composition can include a filler to impart radiopaque, radiolucent, and/or nonradiopaque visual characteristics to the composition. The particles can include organic materials and inorganic materials. Examples of organic fillers include pulverized polycarbonates, polyepoxides, and the like. Additionally, polymeric particles or microbeads comprised of homopolymers or heteropolymers of the monomers described herein can be used as organic fillers. Also, mixtures of fillers can be used.

Examples of inorganic fillers are naturally-occurring or synthetic materials such as quartz, nitrides (e.g., silicon nitride), colloid silica, feldspar, borosilicate, kaolin, talc, ytterbium trifluoride, and various glasses. The glasses can be comprised of, for example, silicon (Si), cerium (Ce), antimony (Sb), tin (Sn), zirconium (Zr), strontium (Sr), barium (Ba), aluminum (Al), zinc (Zn), and the like. More particularly, the glasses can be oxides of these materials.

In one embodiment, the composition can include a filler at a preferred range of from about 0% to about 90% by weight, more preferred range of from about 0% to about 50% by weight, and most preferred range of from about 0% to about 25%. The filler can be comprised of particles having a preferred diameter range of from about 0.005 micrometers to about 50 micrometers, more preferred range of from about 0.5 micrometers to about 25 micrometers, or most preferred range of from about 1 micrometer to about 10 micrometers. For alternative embodiments it may be more preferable for the fillers having an average particle size of from about 0.005 micrometers to about 2 micrometers can be used. However, larger or smaller particles sizes can be used. Additionally, x-ray opaque fillers having particles sizes less than 5 micrometers such as ytterbium trifluoride and the like can impart beneficial characteristics to the tooth.

In order to enhance the bond between the filler and the dental composition, a coupling agent can optionally be used. Examples of coupling agents can include, without limitation, gamma-methacryloxypropyltrimethoxysilane, gamma-mercaptopropyltriethoxysilane, beta-(3,4-epoxycyclohexyl)-ethyltrimethoxysilane, gamma-glycidoxypropyltrimethoxysilane, and the like.

Various other additives can be included within the dental composition in accordance with the present invention. These additives can include stabilizers, UV absorbers, polymerization accelerants, polymerization inhibitors, dyes, pigments, and lubricants. Additionally, the dental compositions can include flavorants, anti-microbials, fragrance, viscosity modifiers, rheology modifiers, fluoride releasing materials, and plasticizers.

Another embodiment of the present invention provides a method of performing a dental procedure. The method includes applying a dental composition to a tooth, where the dental composition is comprised of at least one polymerizable resin and at least two different types of color changing materials. Additionally, the method includes exposing the dental composition to a first color changing stimulus so that the color of the dental composition changes from blending with the tooth to a first color contrasting with the tooth. Optionally, the method can also include exposing the dental composition to a second color changing stimulus so that the dental composition changes to a second color contrasting with the tooth. Thus, the method includes using a dual color changing dental composition to temporarily change the color of the dental composition for use in dental diagnostics.

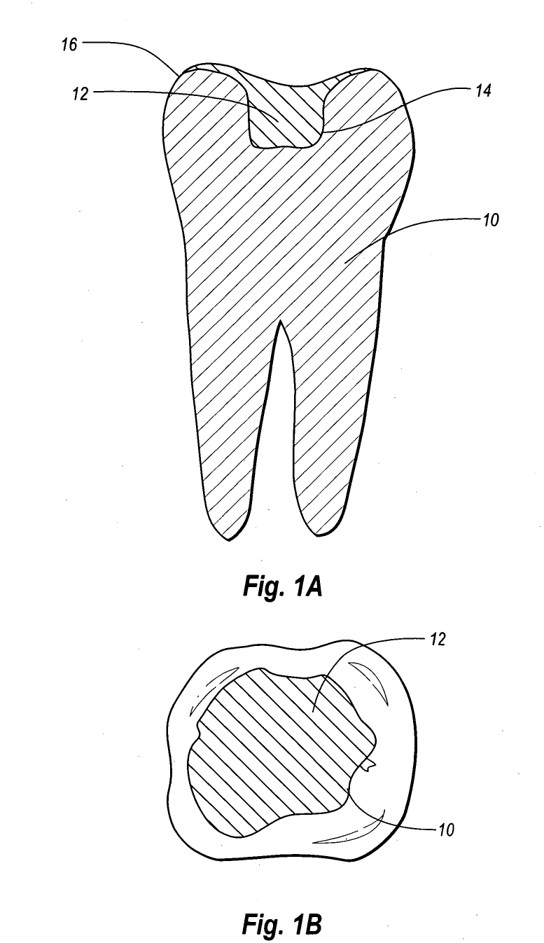

FIGS. 1A and 1B depict an embodiment of the present invention that includes a tooth 10 having a dual color changing dental composition 12 applied thereto. More particularly, during a dental procedure the tooth 10 has a cavity 14 that has been prepared by a dental professional. As such, the dual color changing dental composition 12 can be configured into a filling 12 for use in treating the cavity 14. After the cavity 14 has been filled, the filling 12 can be exposed to a first color changing stimulus for a sufficient duration in order to change the color of dental composition from blending with the tooth to a first contrasting color. Additionally, if needed, the dental composition can be exposed to a second color changing stimulus for a sufficient duration to effect a second color change. In some instances the dental professional may determine that the tooth 10 has a superfluous portion 16 of dental material.

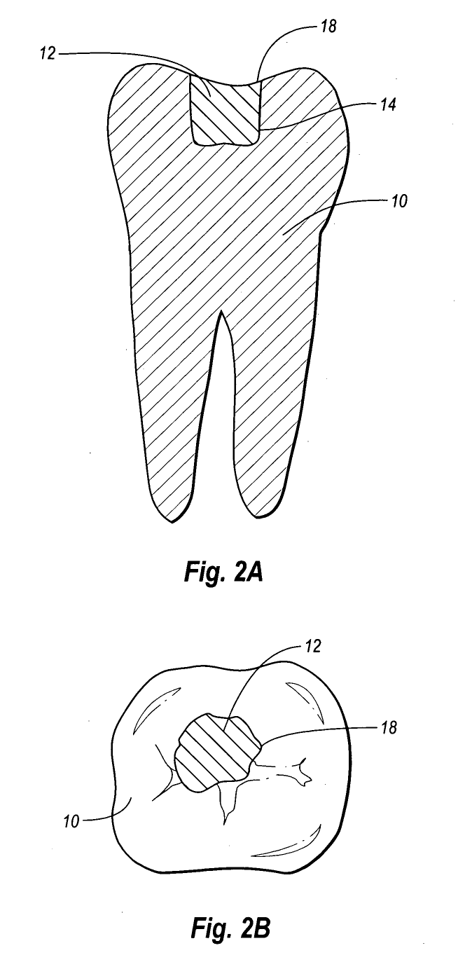

In another embodiment of the present invention depicted in FIGS. 2A and 2B, the dental professional can remove the superfluous portion (as depicted in FIGS. 1A and 1B) from the tooth 10 during the dental procedure. Since the first color change and/or second color change can be visualized prior to curing, the superfluous portion can be easily removed prior to polymerization of the resin. Alternatively, the superfluous portion can be removed after the resin has been cured on the tooth 10. This can be done by grinding or other procedures known and practiced by dental professionals. As such, the tooth 10 can be treated so that the filling 12 fills the cavity 14 to conform to typical dental standards. After this is complete, the dental professional can visualize the boundary 18 between the filling 14 and the tooth by changing the color of the dental composition in order to make sure the treatment is sufficient.

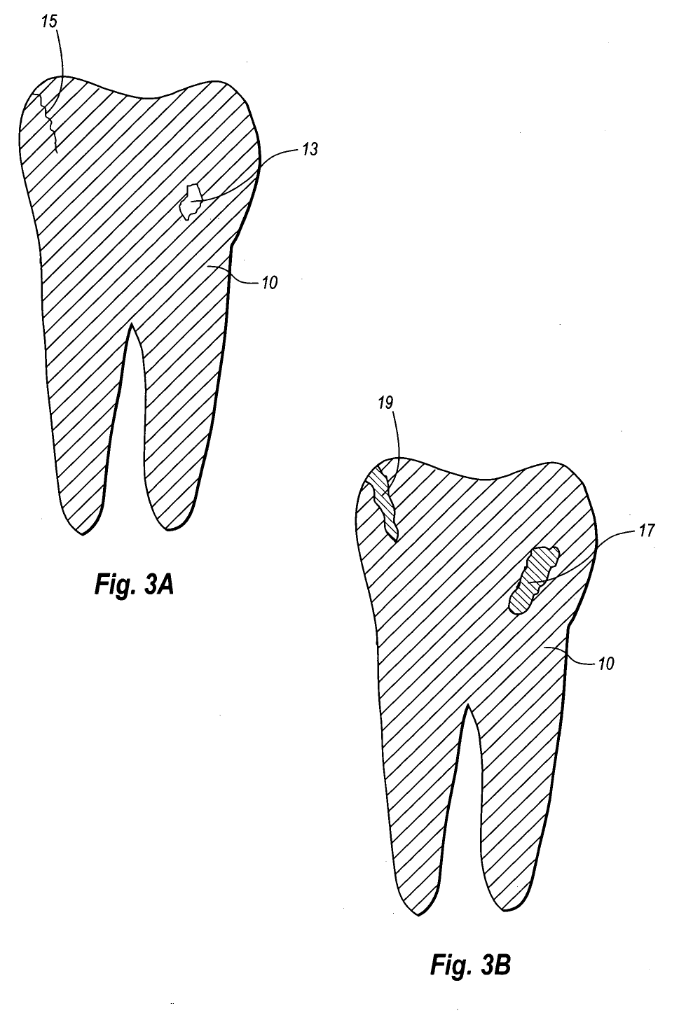

Additionally, FIGS. 3A and 3B depict another embodiment of the present invention that includes repairing a tooth 10 having a pit 13 and fissure 15. During a dental procedure a dental professional can identify the location of a pit 13 or fissure 15 in a tooth 10. Accordingly, the dual color changing dental composition of the present invention can be configured into a sealant. The dental professional can apply the pit sealant 17 over the pit 13. Also, the dental professional can apply the fissure sealant 19 to the tooth 10 so as to seal the fissure 15. In any event, the dental professional can expose the pit sealant 17 and/or the fissure sealant 19 to a first and/or second stimulus to temporarily change the color of the sealant.

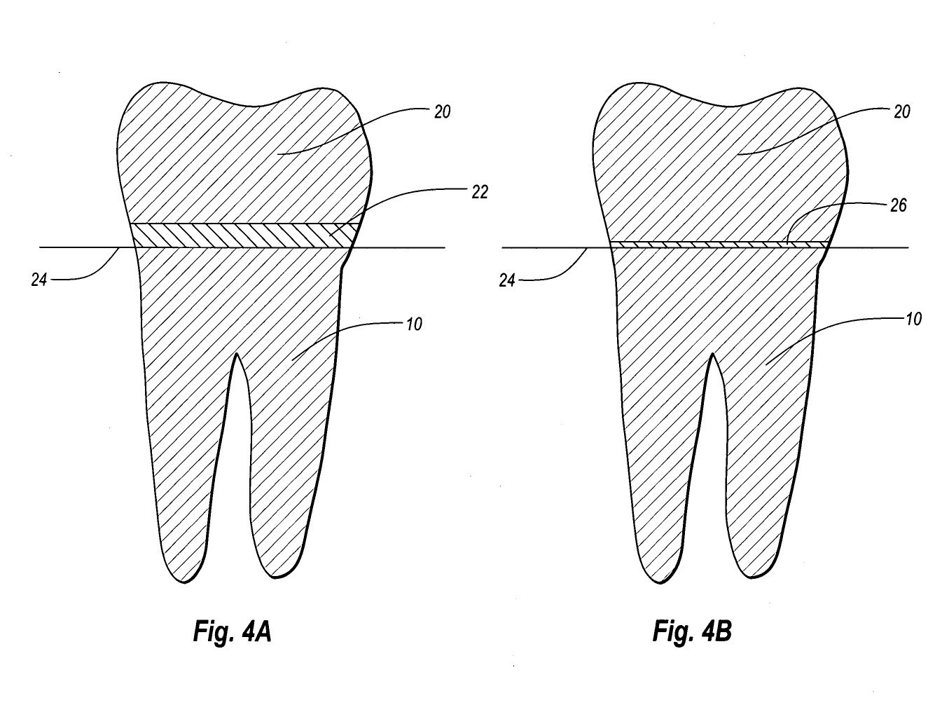

FIGS. 4A and 4B depict another embodiment of the present invention that includes affixing a crown 20 to the tooth 10 during a dental procedure. Accordingly, a kit including a polymerizable resin composition and at least two different types of color changing materials are admixed and configured into a prosthetic adhesive 22. The adhesive 22 is applied such that it is between the tooth 10 and the crown 20 when the crown 20 is being positioned onto the tooth 10, as depicted in FIG. 4A. Usually, the crown 20 will be affixed so that it is above or at the gum line 24. Alternatively, the dental prostheses may be comprised of at least two different types of color changing materials.

After the crown 20 is affixed to the tooth, as depicted in FIG. 4B, a visible boundary 26 may exist. The visible boundary 26 may be observed to distinguish the crown 20 from the tooth 10. In order to better distinguish the crown 20 from the tooth 10, some of the adhesive may be present at the visible boundary 26. As such, the visible boundary 26 may be capable of changing color. Additionally, if any superfluous prosthetic adhesive is forced out from between the crown 20 and the tooth, it may be removed before or after curing.

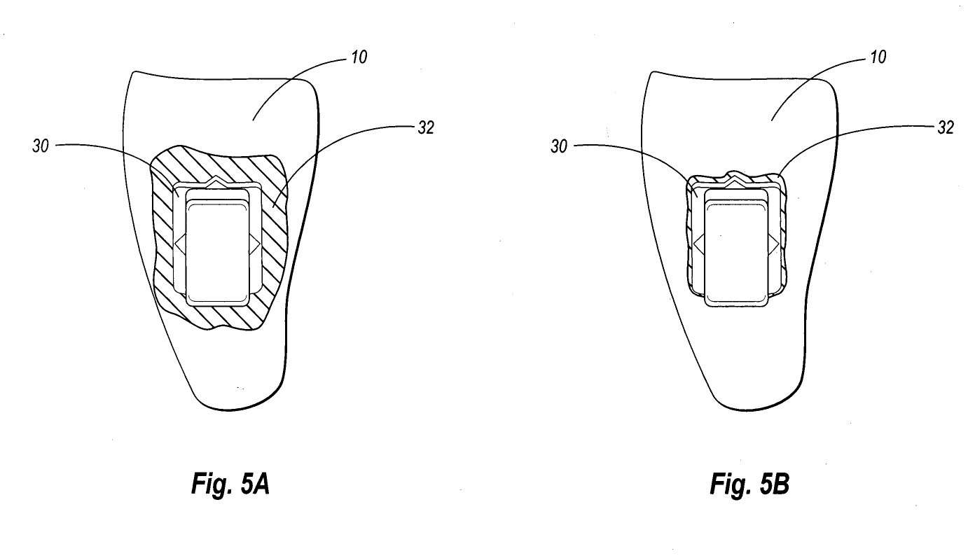

Another embodiment of the present invention includes affixing an orthodontic bracket 30 to a tooth 10, as depicted in FIGS. 5A and 5B. As such, the dental procedure can include applying a dual color changing bracket adhesive 32 to the tooth 10. Alternatively, the bracket adhesive 32 can be applied directly to the bracket 30. The bracket 30 is then applied to the tooth 10, and positioned into a correct location. The adhesive 32 can be exposed to a first and/or second color changing stimulus so that a first and/or second color change can be observed. Accordingly, the dental: professional can determine if there is any extraneous or superfluous adhesive 32 around the bracket 30, as depicted in FIG. 5A. As depicted in FIG. 5B, any superfluous adhesive 32 can be removed before or after curing.

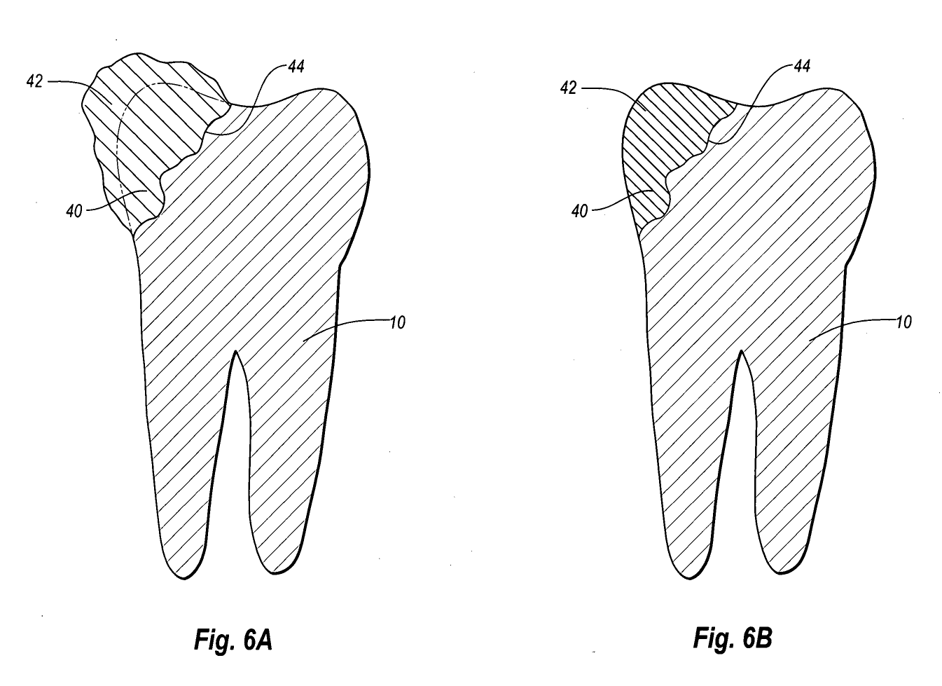

FIGS. 6A and 6B depict another embodiment of the present invention that includes applying a dental restorative composition 40 to the tooth 10. During the dental procedure, a dual color changing dental restorative composition 40 can be applied to the tooth 10. However, it is possible that too much of the restorative composition 40 can be present on the tooth 10 so that a superfluous portion 42 needs to be removed, as depicted in FIG. 6A. In order to facilitate determining how much needs to be removed, the dental professional can expose the restorative composition 40 to a color changing stimulus so that it will change colors. This can also aid in identifying the boundary 46 between the restorative composition 40 and the tooth 10.

Accordingly, it may be determined that a superfluous portion 42 of the restorative composition 40 needs to be removed. As such, the dental procedure can include shaping the restorative composition 40 into a dental restoration 46 as depicted in FIG. 6B. The shaping can be preformed before the restorative composition 40 has been cured. Alternatively, the shaping can be performed after the restorative composition 40 has been cured into a hardened dental restoration 46.

After the dental diagnostic procedure has been completed for any methods of dental procedures that utilize a dental composition including at least two color changing materials, the dental professional can terminate the process of exposing the dental composition with a color changing stimulus. Consequently, the dental composition can then convert back to blending with the patient's teeth. Thus, the dental composition can reversibly change colors for use in dental diagnostics, and then return to the original teeth-blending color.

Subsequent to any of the procedures described, a dental professional may need to distinguish between a dental composition and the tooth after the composition has been in place for some time. Accordingly, the dental professional can expose the dental composition to a first color changing stimulus so that a first color change can be visualized. Additionally, the dental professional can expose the dental composition to a second color-changing stimulus so that a second color change can be visualized. With the aid of the first and/or second color change, the boundary between the old dental composition and the tooth can be determined. For example, if the dental restorative depicted in FIG. 6B needs to be removed and/or replaced, a dual color changing dental composition can be used to enable the dental professional to remove the dental restorative without removing too much of the tooth. Thus, the removal can be performed until the tooth does not exhibit a first color change and/or a second color change.

In accordance with a method for performing a dental procedure described herein, when at least one of the color changing materials is a fluorescent material the color changing stimulus can be a light source such as a UV-light source. As such, the color change can be induced by irradiating the dental composition with a light source so that the fluorescent color change can be observed. Additionally, the color change can be reversed so that the dental composition is the original color by removing the light source.

Also, when the color changing material is a photochromic material, the color changing stimulus can include irradiating the dental composition with a light source, were the wavelength of the light, the intensity of the light, and the duration required to effect the color change can be varied in accordance with the photochromic material. Accordingly, the dental composition can be formulated so that a certain light source stimulus can be used. The dental composition can change back to the original color when the light source is removed. As such, a dental professional can change the color of the dental composition for a short period of time while performing dental diagnostics.

In alternative embodiments, when the color changing material is a thermochromic material, the color changing stimulus can be either heating or cooling the dental composition so that the temperature changes from normal oral temperatures. As used herein, heating includes increasing the temperature of the dental composition to a temperature above about 37° C., and cooling includes decreasing the dental composition to a temperature below about 37° C. However, it may be possible to have a dental composition that will change to one color when heated, and then change to another color when cooled. After the dental diagnostics have been performed, the temperature can be changed back to about 37° C. so that the dental composition can again blend with the surrounding teeth.

In another embodiment, when at least one of the color changing materials is a phosphorescent material the color changing stimulus can be a light source that emits any wavelength of visible or UV-light. Accordingly, the light source can emit any wavelength of light so long as it energizes the phosphorescent material to a level that enables phosphorescent luminescence to be emitted from the dental composition after the intensity of the light is sufficiently decreased or the light source is removed. Additionally, the method can be further comprised of removing the light and/or decreasing the intensity of the light source so that the phosphorescence being emitted from the dental composition can be visualized.: Alternatively, the phosphorescence can be observed while under UV-light. This can enable the dental professional to distinguish between the tooth and the dental composition applied thereto.

The present invention is further exemplified in the following examples, which are offered by way of illustration and are not intended to limit the invention in any manner.

### EXAMPLE 1

A light-curable dental composition having dual color changing capabilities is formulated for use as a filling 7-amino-4-methylcoumarin at 0.1% by weight is mixed with a ZnS:Cu complex at 2% by weight of the filling composition. Additionally, 22% by weight Bis-GMA is also admixed therewith. The mixture is processed with 50% by weight barium silicate glass powder, 5% by weight barium fluorosilicate glass powder, 5% by weight pyrogenic silica, and 15% by weight ytterbium trifluoride to produce a homogeneous composite. Additionally, 0.7% by weight camphorquinone, 0.1% by weight hydroquinone monoethyl ether, and 0.1% by weight ethyl-4-dimethylaminobenzoate are admixed into the homogeneous composite.

The dual color changing mixture is applied to an artificial tooth as a filling, and cured by 4 minutes of irradiation with light at a wavelength of 400-500 nm. When the filling material is exposed to UV-light, it is expected that the filling will emit a violet-blue fluorescence. After the UV-light is extinguished, it is expected that the filling material will display green phosphorescence. During the fluorescence and phosphorescence, it is expected that a boundary will be observed between the filling material and the artificial tooth.

### EXAMPLE 2

A dual color changing light-curable orthodontic bracket adhesive is prepared in accordance with the present invention. 7-dimethylamino-4-trifluoromethylcoumarin at 0.2% by weight and a TC-3 Redo (thermochromic material; Artemis US Importers, LLC) at 0.2% by weight of the adhesive composition are admixed with 88% by weight Bis-GMA. The mixture is then processed with 10.7% by weight silanized barium glass powder. Additionally, 0.5% by weight camphorquinone, 0.2% by weight hydroquinone monoethyl ether, and 0.2% by weight ethyl-4-dimethylaminobenzoate are admixed into the composition to form a bracket adhesive.

The dual color changing bracket adhesive is applied to an artificial tooth, and cured by irradiation with light at a wavelength of 470 nm for 3 minutes. The cured bracket adhesive is exposed to UV-light at 37° C., which will stimulate a green fluorescence to emit from the dental composition. It is expected that the green florescent material will be distinguishable from the artificial tooth. After the UV-light is removed, the bracket adhesive is placed in white light and cooled to below 25° C., which will stimulate the dental composition to display a red color. It is expected that the red color will also be distinguishable from the artificial tooth.

### EXAMPLE 3

A dual color changing light-curable dental sealant is prepared in accordance with the present invention. Oxazolidinospirooxazine at 3% by weight and 7-amino-4-methylcoumarin at 0.2% by weight of the dental sealant are admixed into 60% by weight Bis-GMA and 36% by weight triethylene glycol dimethacryate. Additionally, 0.4% by weight camphorquinone, 0.2% by weight hydroquinone monoethyl ether, and 0.2% by weight ethyl-4-dimethylaminobenzoate are admixed with the composition to form a dual color changing dental sealant.

The dual color changing dental sealant is applied to an artificial tooth, and cured by irradiation with light at a wavelength of 400-500 nm for 3 minutes. The cured dental sealant on the artificial tooth is exposed to white light, which will cause the color of the composition to become red. It is expected that the red composition will be distinguishable from the artificial tooth. The dental sealant is then exposed to UV-light, which will cause the dental sealant to emit a violet-blue florescence. It is expected that the violet-blue fluorescence will also be distinguishable from the artificial tooth.

The present invention may be embodied in other specific forms without departing from its spirit or essential characteristics. The described embodiments and examples are to be considered in all respects only as illustrative and not restrictive. The scope of the invention is, therefore, indicated by the appended claims rather than by the foregoing description. All derivatives which come within the meaning and range of equivalency of the claims are to be embraced within their scope.

What is claimed is:

1. A dental composition that blends with a color of a person's tooth and is capable of reversibly changing color for use in dental diagnostics, the dental composition comprising:
at least one polymerizable resin in a dental composition configured for placement on a person's tooth; and
at least two different types of color changing materials admixed into the dental composition, the dental composition being capable of blending with the person's tooth, being capable of a first reversible color change when exposed to a first color changing stimulus, and being capable of a second reversible color change when exposed to a second color changing stimulus.

2. A dental composition in accordance with claim 1, further comprising a free radical polymerization initiator selected from the group consisting of a photo-initiator, heat-initiator, chemical-initiator, and combinations thereof.

3. A dental composition in accordance with claim 1, further including a got filling material.

4. A dental composition in accordance with claim 1, wherein the at least two different types of color changing materials are selected from the group consisting of a fluorescent material, a phosphorescent material, a photochromic material, and a thermochromic material.

5. A dental composition in accordance with claim 4, wherein at least one of the at least two different color changing materials is a fluorescent material.

6. A dental composition in accordance with claim 5, wherein at least one of the at least two different color changing materials is a photochromic material.

7. A dental composition in accordance with claim 5, wherein at least one of the at least two different color changing materials is a thermochromic material.

8. A dental composition in accordance with claim 1, wherein at least one of the at least two different types of color changing materials is encapsulated in a glass or a microbead.

9. A two-part composition for use in making a dual color changing dental composition, the two-part composition comprising:
at least one dental composition configured for placement onto a person's tooth; and
at least two different types of color changing materials for combining with the at least one dental composition, wherein the at least one dental composition combined with the at least two different types of color changing materials is capable of blending with the person's tooth, capable of a first reversible color change when exposed to a first color changing stimulus, and capable of a second reversible color change when exposed to a second color changing stimulus.

10. A two-part composition in accordance with claim 9, wherein the at least one dental composition includes a polymerizable resin.

11. A two-part composition in accordance with claim 10, wherein the at Feast one dental composition further comprises a free radical polymerization initiator selected from the group consisting of a photo-initiator, heat-initiator, chemical-initiator, and combinations thereof.

12. A two-part composition in accordance with claim 9, wherein the at least one dental composition includes a filling material.

13. A two-part composition in accordance with claim 9, wherein the at least two different types of color changing materials are selected from the group consisting of a fluorescent material, a phosphorescent material, a photochromic material, and a thermochromic material.

14. A two-part composition in accordance with claim 13, wherein the at least two different types of color changing materials are comprised of a fluorescent material and one of a photochromic material and a thermochromic material.

15. A two-part composition in accordance with claim 13, wherein the at least one of the at least two different types of color changing materials is encapsulated in a glass or a microbead.

16. A kit in accordance with claim 9, wherein the at least one dental composition is formulated into a form selected from the group consisting of a powder, liquid, suspension, dispersion, emulsion, paste, gel, cream, and solid.

17. A method of performing a dental procedure, the method comprising:
applying a dental composition to a tooth, the dental composition being comprised of at least one polymerizable resin and at least two different types of color changing materials, wherein the dental composition is capable of blending with the tooth; and
exposing the dental composition to a first color changing stimulus to cause a first reversible change in the color of the dental composition.

18. A method in accordance with claim 17, further comprising polymerizing the dental composition on the tooth.

19. A method in accordance with claim 17, further comprising exposing the dental composition to a second color changing stimulus to cause a second reversible change in the color of the dental composition.

20. A method in accordance with claim 17, wherein the at least two different types of color changing materials are selected from the group consisting of a fluorescent material, a phosphorescent material, a photochromic material, and a thermochromic material.

21. A method in accordance with claim 17, further comprising removing a superfluous portion from the dental composition.

22. A method in accordance with claim 17, further comprising affixing one of a dental prosthesis and an orthodontic bracket to the tooth.

23. A method in accordance with claim 17, further comprising shaping the dental composition into a dental restoration.

24. A method in accordance with claim 17, further comprising removing the dental composition from the tooth.
